(19) United States
(12) Patent Application Publication
(10) Pub. No.: US 2018/0238852 A1
(43) Pub. Date: Aug. 23, 2018

(54) METHOD AND DEVICE FOR DETERMINING THE TEMPERATURE AND HUMIDITY OF A BUILDING

(71) Applicant: PUCHEGGER U. JILG PARKETT GROSS U. EINZELHANDELS GES,M,B.H., Krumbach (AT)

(72) Inventors: Helmut JILG, St. Christophen (AT); Alfred PUCHEGGER, Krumbach (AT)

(21) Appl. No.: 15/752,136

(22) PCT Filed: Aug. 11, 2016
(86) PCT No.: PCT/AT2016/060028

§ 371 Date: Feb. 12, 2018

(30) Foreign Application Priority Data
Aug. 13, 2015 (AT) A 50718/2015

(51) Int. Cl. G01N 33/38; G01N 25/56

(52) U.S. Cl. CPC G01N 33/383; G01N 25/56

(57) ABSTRACT

The invention relates to a method and a device for determining the temperature and moisture of a structure, having at least one temperature sensor and at least one moisture sensor, wherein a water-vapor diffusion-impermeable volume is formed on the surface of the structure, and, after reaching a state of equilibrium, the temperature and moisture is measured with the at least one temperature sensor and the at least one moisture sensor in the water-vapor diffusion-impermeable volume, and used for determining the temperature and moisture in the structure. According to the present invention, the water-vapor-diffusion equivalent air layer thickness of the water-vapor diffusion-impermeable volume is at least 1000 m, and the temperature and moisture of the ambient air are measured.

The invention relates to a method for determining the temperature and moisture of a structure, having at least one temperature sensor for measuring the temperature of the structure and at least one moisture sensor for measuring the moisture of the structure, wherein a water-vapour diffusion-impermeable volume is formed on the surface of the structure, and, after reaching a state of equilibrium, the temperature and moisture is measured with the at least one temperature sensor and the at least one moisture sensor in the water-vapour diffusion-impermeable volume, and are used for determining the temperature and moisture in the structure.

Furthermore, the invention relates to a device for determining the temperature and moisture of a structure, having at least one temperature sensor and at least one moisture sensor, wherein a water-vapour diffusion-impermeable volume is formed on the surface of the structure, in which water-vapour diffusion-impermeable volume the at least one temperature sensor and the at least one moisture sensor are arranged and connected to a microprocessor, so that after reaching a state of equilibrium the temperature and moisture in the water-vapour diffusion-impermeable volume are used as the temperature and moisture in the structure.

In the case of various structures, in particular screeds, the measurement of the so-called microclimate, in particular the temperature and moisture is important or even mandatory in order be able to determine the time of any subsequent work, in particular the time of covering the screed with a floor covering (so-called readiness for laying), without damaging the floor covering due to inadmissibly high moisture values of the screed. In the case of other structures, such as e. g. facades, the condition of the facade, especially its moisture can also be important for any subsequent insulation or painting work.

Previous methods and devices for determining the temperature and moisture of a structure are associated with a partial destruction of the structure by applying boreholes or the like. For example, the DE 36 41 875 A1 describes a method and device for the continuous determination of temporally variable moisture distributions in components, wherein a measuring rod having several temperature and moisture sensors is inserted into a borehole in the component.

The EP 0 901 626 B1 also describes a method and a device for measuring the moisture of structure materials, with corresponding sensors being inserted in a borehole in the structure material.

For the moisture measurement in screeds to determine the readiness for laying, the so-called CM method has established itself as a standard, which calls for the arrangement of boreholes in the screed. Hence, such methods are relatively time-consuming and expensive and moreover often inaccurate.

After covering a screed with a flooring, in particular a wooden flooring, it is possible to record the ambient parameters in a non-invasive manner in order to be able to determine the causes of any possible damage to the floorings in a more reliable way. For example, EP 1 817 529 B2 describes such a device and a method of detecting ambient parameters in floor coverings and a floor covering with such a device. Here, for example, it is possible to determine inadmissibly high temperatures or moisture values in the floor and to prevent subsequent costly expert opinions to find out the causes. However, an inadmissibly high moisture of the screed cannot be determined by this method, or only too late, i.e. after the flooring has been laid.

Methods for determining the temperature and moisture of a structure of the given kind in a non-destructive way are known e. g. in the DE 10 2006 055 095 B3 and DE 10 2005 017 550 A1. From the DE 34 09 453 A1 a method for the non-destructive determination of the moisture of bodies made of solid, porous materials has become known, wherein the measuring air is transported via a drain and supply line to a moisture measuring device and back again. This constitutes a relatively high measuring effort.

Non-destructive moisture measurements, e. g. by measuring the impedance of the structure frequently involve inadmissible inaccuracies.

The object of the present invention is to provide a method and device for determining the temperature and moisture of a structure, in particular screed, which can be carried out as simply, quickly and reliably as possible and has a design which is as simple, inexpensive and robust as possible. Disadvantages of known methods and devices should be avoided or at least reduced.

With regard to the method, the object according to the invention is achieved in the water-vapour diffusion equivalent air layer thickness (sd-value) of the water-vapour diffusion-impermeable volume is at least 1000 m and the temperature and moisture of the ambient air are measured. The method is characterized by the formation of a water-vapour diffusion-impermeable volume which has a certain minimum density and adjoins the structure to be inspected, whereby after a certain period of time a state of equilibrium is achieved by stopping the water-vapour diffusion. As a result, the temperature and moisture values measured in the water-vapour diffusion-impermeable volume can be equated with the ones measured in the structure to be inspected, such as screed. Thus, a relatively fast, clean, non-destructive and reliable determination of the temperature and moisture values in the structure is possible. The moisture measurement usually is a measurement of the relative air moisture. A certain minimum impermeability of the water-vapour diffusion-impermeable volume is required to achieve the state of equilibrium in the shortest possible time. The impermeability can be achieved relatively quickly and easily by using suitable adhesives. When selecting the adhesives care must be taken to ensure that in addition to the water-vapour impermeability and good adhesion properties on the surface of the structure a relatively easy removal from the structure is also possible subsequently. The additional measurement of the temperature and moisture of the ambient air provides important additional information about factors that influence the drying process of the structure.

For further processing and later use of the data, it is preferred to store the measured values of the temperature and moisture. Thus, the measured values are available for later documentation or further processing.

The measured values of the temperature and moisture can also be transmitted to a receiver. In this way, the measured values can be transmitted continuously or at any time to the processing points. Transmission can take place to receivers in the immediate vicinity of the device, for example via Bluetooth® or NFC (Near Field Communication) or also via mobile phone or Internet technologies to receivers situated farther away.

The water-vapour diffusion equivalent air layer thickness (sd-value) of the water-vapour diffusion diffusion-impermeable volume is preferably at least 10000 m.

The water-vapour diffusion-impermeable volume can easily be formed by gluing a sealed chamber onto the surface of the structure.

When the position of the water-vapour diffusion-impermeable volume is determined, on the one hand, the measuring point can be defined clearly and, on the one hand, any unlawful change of the position and, for example, theft of the device can be indicated.

The object according to the invention is also achieved by an above-cited device for determining the temperature and moisture of a structure, wherein the water-vapour diffusion equivalent air layer thickness (sd-value) of the water-vapour diffusion-impermeable volume is at least 1.000 m, and at least one temperature sensor for measuring the ambient temperature and at least one moisture sensor for measuring the ambient moisture is provided and connected to a microprocessor. Such a device is relatively simple and inexpensive and can be manufactured at a small size. With regard to any further advantages, reference is made to above description of the method according to the invention for determining the temperature and moisture of a structure. As mentioned above already, at least one temperature sensor for measuring the ambient temperature and at least one moisture sensor for measuring the ambient moisture is provided and connected to the microprocessor. These measured values provide important information about the environmental conditions that influence the drying process of the structure.

The water-vapour diffusion-impermeable volume can be limited by a ring, on which ring an adhesive layer is arranged for fixing on the upper surface of the structure. Preferably, the ring has the shape of a circle, but other shapes such as triangular, square or polygonal shapes are also possible. At any point of the water-vapour diffusion-impermeable volume there is an opening in which, at best protected by a membrane, the temperature and moisture sensors are arranged. Separate temperature and moisture sensors or also a combined temperature and moisture sensor can be used.

When the microprocessor is connected to a memory, the measured values are retained for later use or documentation.

When the microprocessor is connected to a transmission device, in particular a mobile radio module, the measured values can be transmitted to certain receivers immediately or later. For example, a GSM (Global System for Mobile Communications), UMTS (Universal Mobile Telecommunications System) or LTE (Long Term Evolution) module can be used in the device to realize a connection to a mobile radio network.

In addition, the microprocessor can be connected to a display in order to be able to display certain operating states or measured values. The display can be realized by one or more LEDs or an LCD display at best combined with an operating facility in the form of a touch screen.

When the microprocessor is connected to an interface, measured values can be transmitted outwardly or even updates can be made on the device. For example, the device can be equipped with a USB (Universal Serial Bus) interface. In addition to wired interfaces, wireless interfaces are also possible.

According to another feature of the invention an anti-theft protection is provided. On the one hand, such anti-theft protection can be achieved mechanically by connecting the measuring device to the structure or screed to be examined, however, which leads to partial destruction or impairment of the structure, for example by providing anchors or hooks. In addition to mechanical anti-theft devices it is also possible to implement electronic anti-theft devices, for example in the form of movement sensors and acoustic and/or optical warning devices. As already mentioned above, an anti-theft protection can also be provided by monitoring the position of the device, and in case of any unforeseen changes of the position an acoustic and/or optical warning or even a message can be sent to a central office (e. g. by SMS, e-mail or the like).

The invention will be illustrated in more detail by means of the enclosed drawings showing embodiments of the invention, in which:

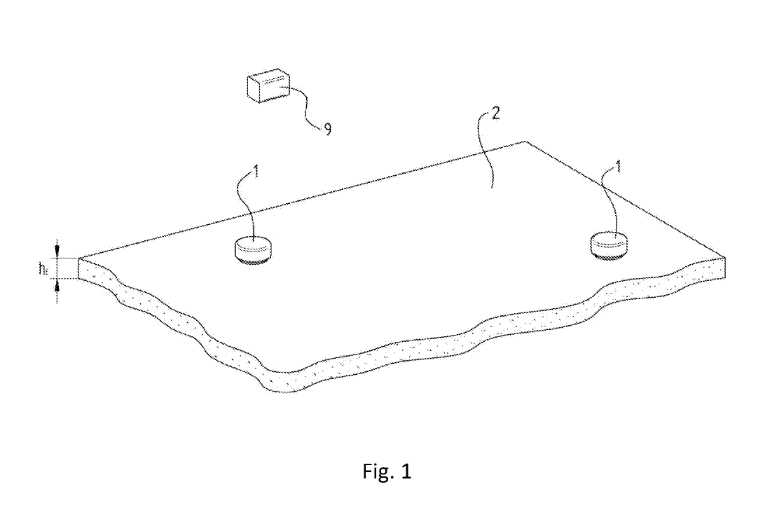

FIG. 1 is a perspective view to illustrate the arrangement of the device according to the invention for measuring the temperature and moisture of a structure in the form of a screed;

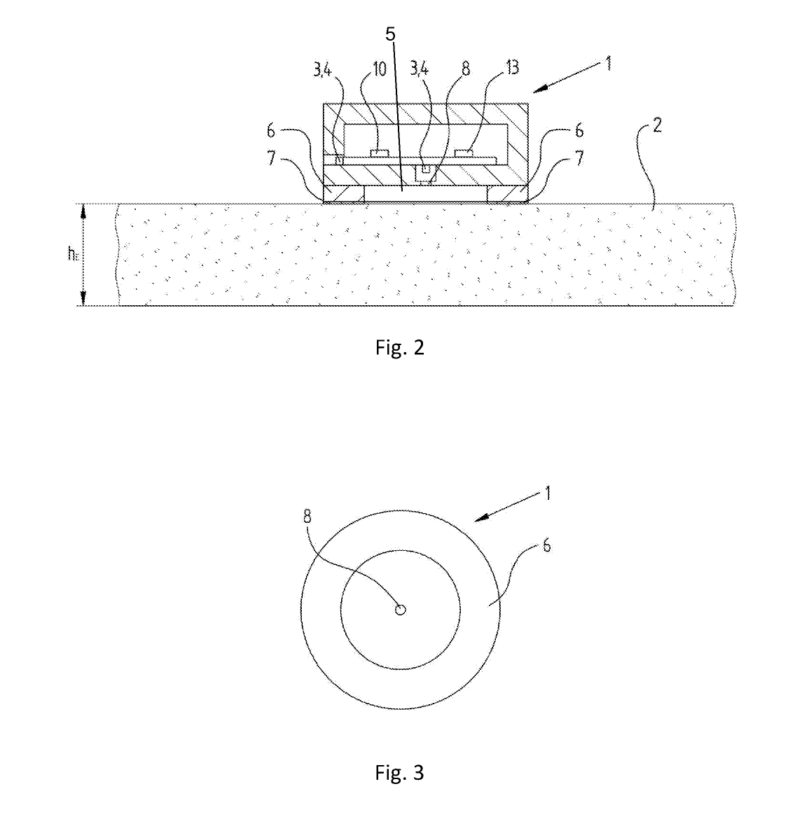

FIG. 2 is a lateral section of an embodiment of device according to the invention for measuring the temperature and moisture of a structure in the form of a screed;

FIG. 3 is a view of the device according to FIG. 2 from below; and

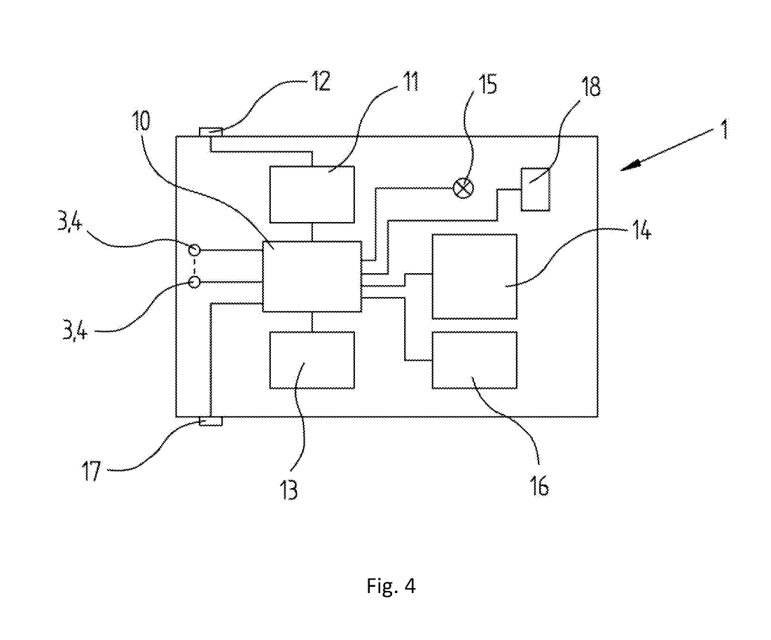

FIG. 4 is a block diagram of a device according to the invention for measuring the temperature and moisture of a structure.

FIG. 1 shows a perspective view to illustrate the arrangement of the device 1 according to the invention for determining the temperature and moisture of a structure 2 in the form of a screed. The structure 2 or the screed, respectively has a certain height h_{E}, on which depends the drying period. The device 1 according to the invention is simply applied to the surface of structure 2 or screed, in particular by gluing, whereby a water-vapour diffusion-impermeable volume 5 is formed, in which a state of equilibrium is created after a certain time t_{GG}, so that the temperature and moisture measured in the water-vapour diffusion-impermeable volume 5 essentially corresponds to the temperature T_{E} and moisture F_{E} of the screed. The time t_{GG} to reach the state of equilibrium mainly depends on the height h_{E} of the structure 2 to be examined and on the size of the water-vapour diffusion-impermeable volume 5. In the case of standard screed heights h_{E} of 5 to 6 cm and a diameter of the water-vapour diffusion-impermeable volume 5 of 1.5 times the screed height h_{E}, i.e. about 9 cm, it is expected that the state of equilibrium will be reached within a few days to one week, for example. However, when the respective temporal characteristics are known, a reliable conclusion on the temperature and moisture in structure 2 can be drawn before the state of equilibrium has been reached by applying appropriate corrections. This means that the temperature and moisture curve of the screed can be determined quickly and easily by using a non-invasive and non-destructive method, thus reliably determining the point in time at which a floor covering can be laid on top of structure 2 or the screed without any problems. Applications on other structures 2, in particular facades for determining the state of the facade, for example to determine the ideal point in time for the application of an insulation or paint layer, are also conceivable.

Several devices 1 for determining the temperature T_{E} and moisture F_{E} can also be placed on the structure 2 or the screed, which can also communicate with each other. In addition, the data of the devices 1 can be forwarded to a receiver 9, from which the measured values can be transmitted to any location, for example in the internet. If a transmission device 14 (see FIG. 4) is arranged in at least one device 1, the measured values can be transmitted directly to a remote receiver 9. FIG. 2 shows a lateral section of an embodiment of a device 1 according to the invention for determining the temperature and moisture of a structure 2 or screed. The device 1 for determining the temperature T_{E} and moisture F_{E} of structure 2 comprises a water-vapour diffusion-impermeable volume 5, which is formed by arranging the device 1 on the surface of structure 2. The water-vapour diffusion-impermeable volume 5, for example, is limited by a ring 6, preferably a circular ring 6, on the side and by the housing of the device 1 on the top surface. Provided a corresponding impermeability of volume 5 is given, a state of equilibrium is reached after a period of time that is largely dependent on the height h_{E} of structure 2 or screed, and the temperature and moisture measured in water-vapour diffusion-impermeable volume 5 can be used to determine the temperature T_{E} and moisture F_{E} in the structure 2 or the screed. To measure the temperature and moisture in the water-vapour diffusion-impermeable volume 5 an opening 8 is arranged in the housing of device 1, behind which the temperature sensor 3 and the moisture sensor 4, at most a combined temperature and moisture sensor is arranged. The temperature sensor 3 and moisture sensor 4 are connected to a microprocessor 10, which evaluates the data accordingly and stores them in a memory 13 at best. Further temperature sensors 3 and moisture sensors 4 are arranged for measuring the ambient temperature T_{U} and the ambient moisture F_{U}, which are connected to the environment via a corresponding opening. The device 1 should preferably be affixed to the surface of structure 2 by means of an appropriate adhesive layer 7, which can be formed by a liquid or pasty adhesive, at best on a carrier layer. The adhesive is selected to achieve the impermeability of the water-vapour diffusion-impermeable volume 5 and to achieve an appropriate adhesion on the structure 2.

FIG. 3 shows a view of the device 1 according to FIG. 2 from below. It is preferred to design the ring 6 for limiting the water-vapour diffusion-impermeable volume 5 in a circular shape, but theoretically it may also have a triangular, square, rectangular shape or have the form of a polygon.

FIG. 4 shows a block diagram of a device 1 according to the invention for measuring the temperature and moisture of a structure 2 or screed. In addition to the microprocessor 10 described above, which is connected to the temperature sensors 3 and moisture sensors 4, and a memory 13, a power supply 11 is provided, which can be formed in particular by corresponding accumulators. The accumulators can be charged by means of a charging plug 12. A transmission facility 14 connected to the microprocessor 10 is used for the preferably wireless transmission of the measured values to corresponding receivers 9. A GPS (Global Positioning System) module 16 connected to the microprocessor 10 can determine the position of device 1 and also store and transmit this information, especially when using a variety of devices 1, together with the measured values of temperature and moisture. Furthermore, an interface 17 can be connected to the microprocessor 10 in order to read out data from the device 1 or to be able to import new data or software updates into device 1 or microprocessor 10. The interface 17, for example, can be formed by a USB interface. A display 15, e. g. LEDs or an LCD display or a touch screen, can be used to output warning signals, operating states or measured values. An anti-theft device 18 can give a visual or audible warning if the position of device 1 is subject to undesirable positional changes.

1. A method for determining temperature and moisture of a structure, having at least one temperature sensor for measuring the temperature of the structure and at least one moisture sensor for measuring the moisture of the structure, wherein a water-vapor diffusion-impermeable volume is formed on a surface of the structure, and, after reaching a state of equilibrium, the temperature and moisture is measured with the at least one temperature sensor and the at least one moisture sensor in the water-vapor diffusion-impermeable volume, and used for determining the temperature and moisture in the structure, wherein the water-vapor diffusion-equivalent air layer thickness of the water-vapor diffusion-impermeable volume is at least 1000 m, and the temperature and moisture of ambient air are measured.

2. The method according to claim 1, wherein the measured values of the temperature and moisture are stored.

3. The method according to claim 1, wherein the measured values of the temperature and moisture are transferred to a receiver.

4. The method according to claim 1, wherein the water-vapor diffusion-equivalent air layer thickness of the water-vapor diffusion-impermeable volume is at least 10000 m.

5. The method according to claim 1, wherein the water-vapor diffusion-impermeable volume is formed by gluing a closed chamber onto the surface of the structure.

6. The method according to claim 1, wherein a position of the water-vapor diffusion-impermeable volume is determined.

7. A device for determining temperature and moisture of a structure, having at least one temperature sensor and at least one moisture sensor, wherein a water-vapor diffusion-impermeable volume is formed on a surface of the structure, in which water-vapor diffusion-impermeable volume the at least one temperature sensor and the at least one moisture sensor are arranged and connected to a microprocessor, so that after reaching a state of equilibrium the temperature and moisture in the water-vapor diffusion-impermeable volume are used as the temperature and moisture in the structure, wherein the water-vapor diffusion equivalent air layer thickness of the water-vapor diffusion-impermeable volume is at least 1000 m, and at least one temperature sensor for measuring ambient temperature and at least one moisture sensor for measuring ambient moisture are provided and connected to the microprocessor.

8. The device according to claim 7, wherein the water-vapor diffusion-impermeable volume is limited by a ring, on which ring an adhesive layer is arranged for fixing on an upper surface of the structure.

9. The device according to claim 7, wherein the microprocessor is connected to a memory.

10. The device according to claim 7, wherein the microprocessor is connected to a transmission facility.

11. The device according to claim 7, wherein the microprocessor is connected to a display.

12. The device according to claim 7, wherein the microprocessor is connected to an interface.

13. The device according to claim 7, wherein an anti-theft device is provided.

14. The device according to claim 7 13, wherein a device connected to the microprocessor for determining a position is provided.

15. The device according to claim 10, wherein the transmission facility is a cellular radio module.

16. The device according to claim 14, wherein the device for determining the position is a GPS (global positioning system) module.
